(19) United States
(12) Patent Application Publication
(10) Pub. No.: US 2019/0290159 A1
(43) Pub. Date: Sep. 26, 2019

(54) ELECTROENCEPHALOGRAPHY SENSORS

(71) Applicant: KOKOON TECHNOLOGY LTD, London (GB)

(72) Inventors: Richard HALL, London (GB); Tim ANTOS, London (GB); Manon Diana Edith VAN THORENBURG, London (GB)

(21) Appl. No.: 16/436,448

(22) Filed: Jun. 10, 2019

Related U.S. Application Data
(63) Continuation of application No. PCT/EP2017/082257, filed on Dec. 11, 2017.

(30) Foreign Application Priority Data
Dec. 12, 2016 (GB) 1621074.2

(51) Int. Cl. A61B 5/0478; A61B 5/00

(52) U.S. Cl. CPC A61B 5/0478; A61B 5/6814; A61B 5/04012; A61B 5/4809; A61B 5/6803; A61B 5/0006

(57) ABSTRACT

Various designs of dry EEG sensor are described to permit movement of the sensor contacts with respect to the device in which the sensor is mounted.

### CROSS REFERENCE TO RELATED APPLICATIONS

This application is a continuation application of International Application PCT/EP2017/082257, which was filed on Dec. 11, 2017, the entire contents of which are incorporated herein by reference. International Application PCT/EP2017/082257 is based upon and claims the benefit of priority of the prior United Kingdom Patent Application No. 1621074.2, filed on Dec. 12, 2016, the entire contents of which are also incorporated herein by reference.

### TECHNICAL FIELD

Embodiments of the present invention generally relate to sensors for use in Electroencephalography (EEG) systems and in particular dry EEG sensors for contacting the head and ear.

### BACKGROUND

Electroencephalography (EEG) measures electrical potential fluctuations on the head (mostly on the scalp) which are caused by brain activity. Analysis of the fluctuations can be utilised to analyse brain activity, for example to determine whether a user is awake or asleep, or their state of sleep.

EEG systems utilise sensors positioned around the head to sense the electrical potential at one or more locations. Non-contact sensors attempt to sense the potential using field sensors, whereas contact-based sensors make an electrical contact to the skin.

Contact-sensors can be categorised as wet or dry, which indicates whether a conductive gel is used to ensure a good electrical contact. Wet sensors are common in clinical applications, but are a disadvantage in consumer devices as they rely on the correct application of the gel. Dry sensors are more convenient for use, but may present difficulties in maintaining the contact stationary with regard to the user's skin.

There is a requirement for an EEG sensor that is comfortable to wear that remains sufficiently static with respect to a users.

The embodiments described below are not limited to implementations which solve any or all of the disadvantages of known systems.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

There is provided an electroencephalography (EEG) sensor, comprising at least one elongate electrical contact for contacting the skin of a user, the at least one electrical contact being mounted in a bobbin; a resilient arm for connection at a proximal end to a device in which the sensor is located, the bobbin being mounted at a distal end of the resilient arm, wherein the resilient arm permits movement of the electrical contacts in an axis substantially aligned with the axis of the at least one elongate electrical contact, and wherein movement between the bobbin and resilient arm permits lateral movement of the tips of the electrical contacts in a plane substantially perpendicular to the axis of the at least one elongate electrical contact.

There is also provided an electroencephalography (EEG) sensor, comprising at least one elongate electrical contact for contacting the skin of a user, the at least one electrical contact being mounted to a bobbin; and a resilient arm for connection at a proximal end to a device in which the sensor is located, the bobbin being mounted at a distal end of the resilient arm via a pivotable joint component permitting pivoting in at least one dimension, wherein the resilient arm permits movement of the electrical contacts in an axis substantially aligned with the axis of the at least one elongate electrical contact, and wherein movement between the bobbin and resilient arm permits lateral movement of the tips of the electrical contacts in a plane substantially perpendicular to the axis of the at least one elongate electrical contact.

24. An electroencephalography (EEG) sensor, comprising at least one elongate electrical contact for contacting the skin of a user, the at least one electrical contact being mounted to a bobbin via a pivotable joint component permitting pivoting in at least one dimension; a resilient arm for connection at a proximal end to a device in which the sensor is located, the bobbin being mounted at a distal end of the resilient arm, and a joint for connecting the proximal end of the resilient arm to a device in which the sensor is located, wherein the resilient arm permits movement of the electrical contacts in an axis substantially aligned with the axis of the at least one elongate electrical contact, and wherein the joint permits movement between the resilient arm and a device in which the sensor is located along a longitudinal axis of the resilient arm.

There is also provided an electroencephalography (EEG) sensor, comprising at least one elongate electrical contact for contacting the skin of a user; a resilient arm for connection at a proximal end to a device in which the sensor is located, the bobbin being mounted at a distal end of the resilient arm, wherein the resilient arm permits movement of the electrical contacts in an axis substantially aligned with the axis of the at least one elongate electrical contact, and wherein movement between the bobbin and resilient arm permits lateral movement of the tips of the electrical contacts in a plane substantially perpendicular to the axis of the at least one elongate electrical contact.

There is also provided an EEG sensor, comprising at least one elongate electrical contact for contacting the skin of a user; a resilient material positioned between the at least one electrical contact and a part of a device in which the EEG sensor is mounted; and a low-friction material positioned between the at least one electrical contact and the resilient material.

A selection of optional features are set out in the dependent claims.

### BRIEF DESCRIPTION OF THE DRAWINGS

Embodiments of the invention will be described, by way of example, with reference to the following drawings, in which:

FIGS. 1 to 4B, and 6 to 7 show various EEG sensors; and

FIG. 5 shows a mechanism to withdraw an EEG sensor.

### DETAILED DESCRIPTION

Embodiments of the present invention are described below by way of example only. These examples represent the best ways of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

Dry sensors rely on a direct contact between the sensor and skin to obtain a signal, and must be held sufficiently stationary on the skin to avoid interference with the signal. Movement of a sensor across the surface of the skin can generate artefact signals many times larger than the actual EEG signals thus making recovery of the actual signals difficult. It is believed that movement on a micron-scale is sufficient to generate artefacts that will interfere with the actual signals. For example, it can be possible to detect the user's heartbeat from artefacts created by skin movement due to the heart beat.

Maintaining static contact is particularly difficult with devices intended to be worn for extended periods of time. For example a device worn overnight must be sufficiently comfortable to allow the user to sleep, while also securing the sensors and preventing movement during sleep. A further challenge is that sensors must reach through hair on the user's head to make contact with the skin.

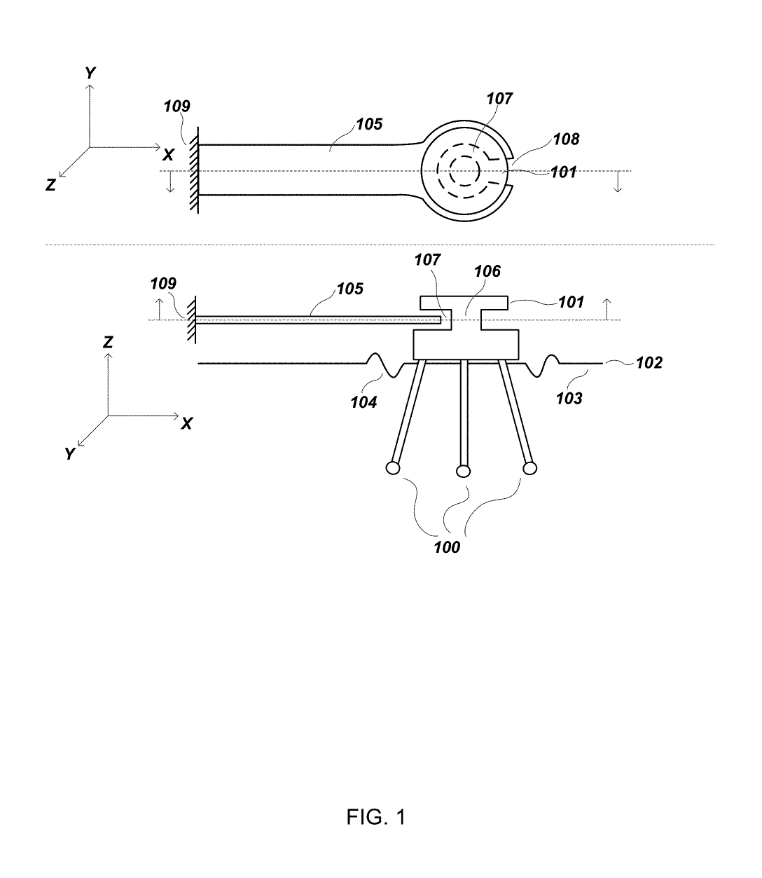

In order to minimise movement between an EEG sensor and the skin of the user a sensor design which can stay sufficiently static, while also applying only a reasonable amount of force to ensure comfort of the user, is required. Furthermore, to sense locations on the user's scalp, the sensor must reach through hair. FIG. 1 shows plan and cross-section diagrams of a sensor arrangement intended to meet these requirements.

Axis X, Y, Z will be used to describe the arrangements shown in the figures. The X/Y plane will be referred to as the lateral plane, and movement in that plane will be described as lateral movement. The Z axis will be described as the vertical axis, and movement in that axis as vertical movement. Terms such as lateral and vertical are used only for description in relation to the figures, and are not descriptive of the actual orientation of the device. In use the sensor is oriented such that the skin of the user is positioned substantially in the X/Y plane.

Electrical contacts 100 are mounted to a bobbin 101 in a substantially rigid arrangement such that the position of the contact tips is maintained relative to each other. The electrical contacts 100 may be formed of any appropriate conductive material, for example copper with a gold coating at the tip for providing good electrical contact to the user's skin. The electrical contacts 100 are in turn connected to electrical circuitry to process the signals detected. Typically the contact is initially connected to an input buffer amplifier (which may be mounted as part of the sensor arrangement). Any electrical connection from the sensor 100 is provided using sufficiently flexible wire to prevent restraining sensor movement. The electrical contacts are elongate to penetrate through a user's hair. For example the electrical sensors may have a length in the range of 5-10 mm.

The electrical contacts 100 protrude through a barrier layer 102 which may be formed as part of the device in which the sensor is positioned. In use the sensor is positioned such that surface 103 of barrier layer 102 faces towards the user and hence the electrical contacts 100 protrude towards the user's skin. Barrier layer 102 is formed with a flexible region 104 (which extends around and encircles the electrical contacts). Flexible region 104 is provided to allow lateral movement of the electrical contacts 100 and bobbin 101. The barrier layer may be formed of a thin flexible material, with folds or concertina sections in the flexible region 104 to permit movement of the electrical contacts 100. For example, the layer may be formed of silicone. In an example silicone with a thickness of less than 0.8 mm may be utilised. In alternative configurations a material with sufficient flexibility to not require concertina sections may be utilised.

Bobbin 101 is attached at a first end of arm 105. In the specific design shown in FIG. 1 the bobbin has a neck portion 106 which fits within an opening 107 in the arm 105. Gap 108 is provided to enable neck portion 106 to be positioned in opening 107. Neck portion 106 has a smaller diameter than opening 107 (but larger than gap 108) such that the bobbin 101 can move laterally relative to the arm 105. In an example the diameter of the opening 107 may be 1 mm larger than the diameter of the neck portion 106 to allow 1 mm of lateral movement in X/Y plane.

The height of the neck portion 106 is substantially the same as the thickness of arm 105 around the opening 107 such that vertical movement is substantially prevented, but lateral movement does not suffer significant friction (the height is shown differently in FIG. 1 for clarity). The arm and bobbin surfaces may be coated to reduce friction. In an example, the bobbin may be formed of Nylon, metal with a PTFE coating, or formed as a composite material with regions of low-friction PTFE in the areas of contact between the bobbin 101 and arm 105.

An opposite end of arm 105 is mounted to the body 109 of the device comprising the sensor. In an example the sensor may be mounted in a pair of headphones, and the arm 105 may be mounted to the headband of those headphones. Arm 105 is flexible in the vertical (Z) direction such that the bobbin 101 can move vertically relative to the body 109 by bending of the arm 105. Arm 105 is, however, substantially stiff in the X/Y plane to prevent lateral movement of the bobbin once it is positioned against the edge of the opening 107. The arm 105 is resilient such that the arm 105 deforms under pressure but provides a force against the surface applying pressure. In use the surface is the user's head against which the electrical contacts 100 are pushed when the device comprising the sensor (for example headphones) is placed on the head. In an example the arm 105 may be formed of annealed spring steel having a thickness of 0.15 mm. The spring-steel arm may have a width of 5 mm and a length of 35 mm, which provides a force of approximately 0.5N at the electrical contacts. Other materials may also be utilised for the arm, with appropriate modification of the parameters to provide a comparable force (or different force as required).

Although not shown in FIG. 1 electrical contacts 100 are connected by suitable wires or electrical connections to an EEG system. The example of FIG. 1 utilises three electrical contacts 100 as this may provide a stable contact to the skin of the user. However, other numbers of contacts may also be utilised. Furthermore the contacts may be electrically connected to a sensor system either collectively or individually.

In a variation of the sensor of FIG. 1, the bobbin 101 may be configured to allow angular movement of the electrical contacts 100 around the Z-axis. This may be achieved by utilising resilient material for the bobbin, or allowing angular movement between the bobbin and arm 105.

The sensor arrangement of FIG. 1 thus provides a force against the user's skin to hold the electrical contacts in position, while allowing movement in the lateral direction. In use this means that when the user's skin moves relative to the device on which the sensor is mounted the electrical contacts move with the user's skin rather than sliding over the surface. Electrical artefacts caused by movement of the sensor over the skin are thus reduced, improving the quality of the signals that can be obtained.

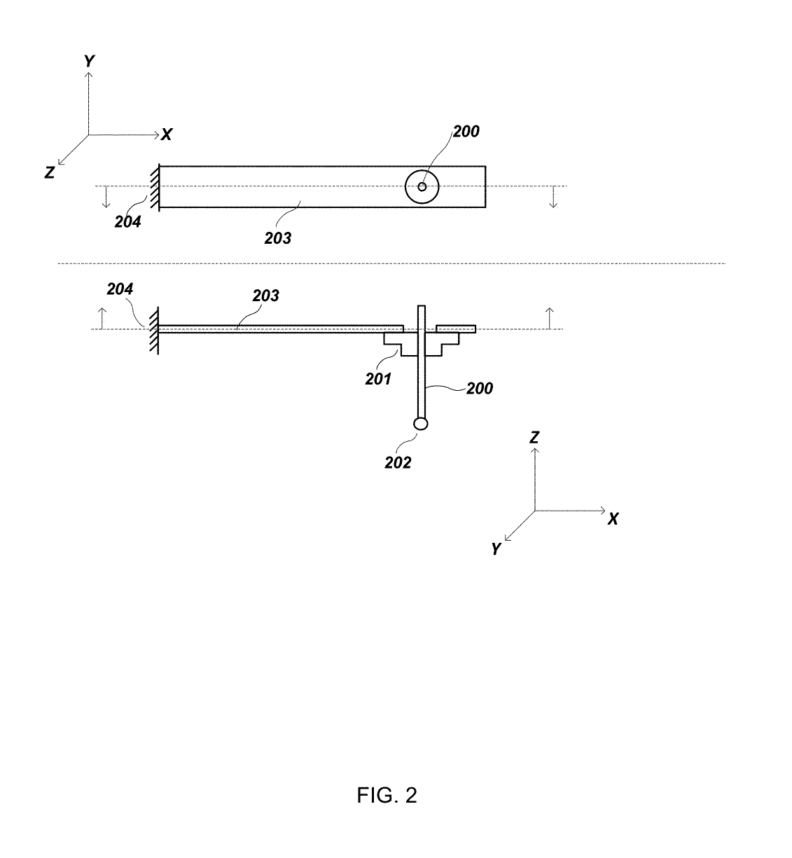

FIG. 2 shows a further example of an EEG sensor for holding an electrical contact static on the skin of a user. Electrical contact 200 is mounted in a flexible bush 201 which permits angular movement of the electrical contact 200 about the X/Y-axis. In an example the flexible bush 201 is formed of rubber. The angular movement provides lateral movement in the X/Y axis at the tip 202 of the electrical contact 200, thus allowing movement between the skin of the user and the device in which the sensor is mounted. In an example the tip may be provided with movement of up to 1 mm about a centre point, or up to 2 mm about a centre point.

The flexible bush 201 is mounted in arm 203, which is resilient, and mounted to the body 204 of the device in which the sensor is positioned. As described in relation to FIG. 1, arm 203 is resilient to movement in the X/Y-axis such that pressure on the electrical contact 200 causes movement of the contact in the Z-axis and the creation of a force against the surface pressing on the contacts.

Bush 201 and electrical contact 200 are mounted to the arm 203 such that the desired movement is possible, and such that the electrical contact 200 is insulated from the arm 203.

As described in relation to FIG. 1, the electrical contact 200 may be formed from gold-plated copper, and the arm 203 may be formed from spring steel. The example values and dimensions provided in relation to FIG. 1 are equally applicable to this example.

A barrier layer may be provided, as described in relation to FIG. 1 with the electrical contact protruding through the barrier layer to contact the user.

In alternative configurations the electrical contact 200 may be curved, for example with an s-bend, which may better enable the contact to penetrate the user's hair.

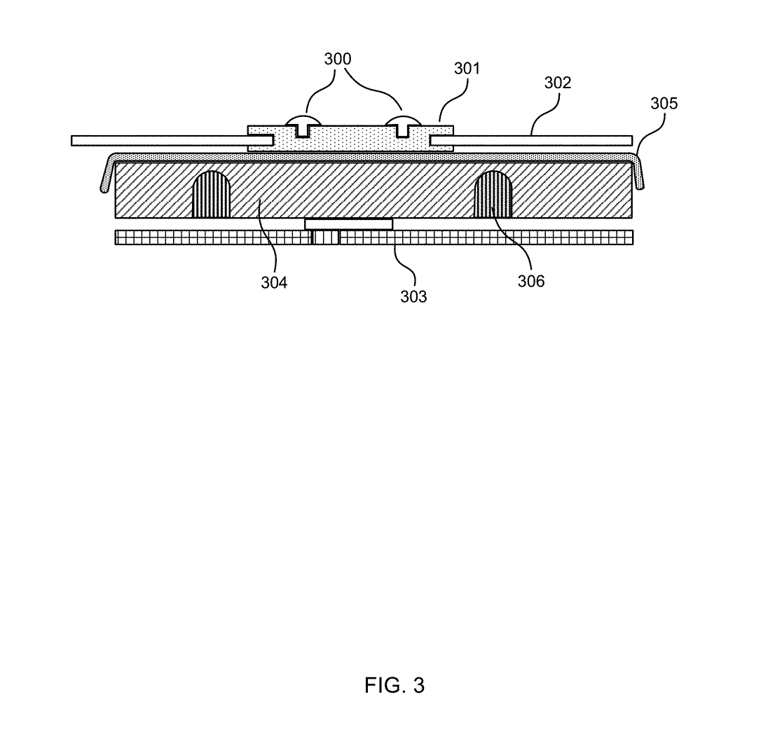

FIG. 3 shows a further example of an EEG sensor. In this example the sensor is not designed to penetrate through significant hair and is intended for use in contacting regions such as the ear.

Electrical contacts 400 are mounted in a bobbin 301, and are electrically connected to the sensing system (not shown) by an appropriate electrical connection. The sensor is mounted in device 303 (of which the figure show a small part in the region of the sensor). The bobbin 301 is mounted in a barrier layer 302, which may be formed of a stretchy fabric which allows relatively free movement of the bobbin in the X/Y plane. As described above the barrier layer may form part of the device. A resilient layer 304, typically formed of foam, is mounted on the device 303. A low-friction material 305 is positioned between the resilient layer 304 and the bobbin 301 such that even when a force is applied to the electrical contacts 300 along the Z-axis the bobbin 301 can still move with little resistance. The resilient layer 304 provides a force against a surface pressing on the electrical contacts 300 and bobbin 301 to maintain the electrical contacts 300 in place

The low-friction material 305 may be provided by the surface of the resilient layer 304 or may be coated onto the surface of the resilient layer 304. Alternative a separate layer may be positioned between the resilient 304 and bobbin 301. The layer 305 may be PTFE (for example PTFE coated tape). Layer 304 may be formed of memory foam or spring type foam material.

A bumper component 306 may be provided to limit movement of the sensor. For example a region of rubber may be provided within the resilient element to stop further movement of the sensor after it has compressed the resilient layer by a defined amount.

The sensor of FIG. 3 can thus maintain a force against the user by the electrical contacts, while also allowing movement of the electrical contact in the X/Y plane to avoid the difficulties of prior sensors.

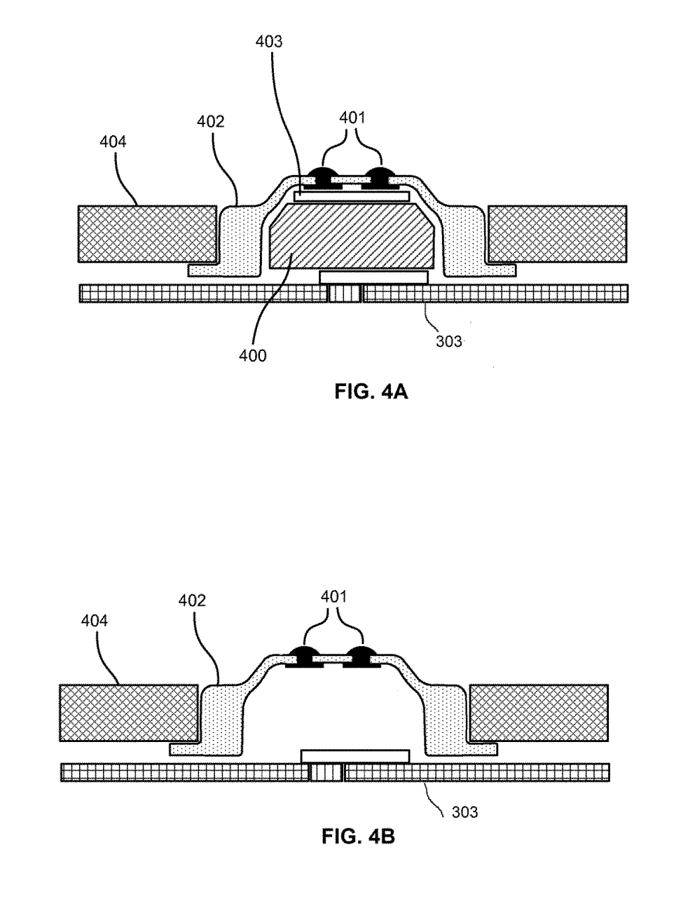

FIG. 4A shows a further example of an EEG sensor. The sensor of FIG. 4 is similar to that of FIG. 3, utilising a resilient layer 400 to provide a force to press the electrical contacts 401 against the skin of a user. In this example the electrical contacts 401 are mounted in a flexible layer 402 which permits movement in all directions. As with the example of FIG. 3 a low-friction layer 403 is provided between the electrical contacts 501 and resilient layer 400 to permit movement of the electrical contacts in the X/Y plane.

The flexible layer 402 may be formed of silicone as described in relation to FIG. 1, and concertina regions may be provided to permit movement of the electrical contacts. A spacer material 404 gives a level surface across the area around and including the sensor.

Resilient layer 400 and low-friction layer 403 may be omitted in certain examples, such as that shown in FIG. 4B, with the flexible layer 402 providing the required support for the electrical contacts 401. FIG. 4B shows a further example of an EEG sensor suitable for mounting on a device 303 such as a set of headphones. The example sensor of FIG. 4B is similar to that of FIG. 4A, but with the resilient layer 400 and low-friction layer 403 omitted and with the flexible layer 402 providing the required support for the electrical contacts 401 on the body of the device 303, and providing a force to press the electrical contacts 401 against the skin of a user when the device 303 is in use (i.e. when the headphones are being worn by a user). In this example the electrical contacts 401 are mounted in a flexible layer 402 which permits movement in all directions, for example, as the skin of the user moves relative to the device. The electrical contacts are configured in some examples to extend through a barrier to make contact with the skin of a user. In examples where the device is a headset of set of headphones, one or more of the electrical contacts may contact the ear of the user. In the example shown in FIG. 4B (and FIG. 4A), the flexible layer 402 may be formed of silicone as described in relation to FIG. 1, and concertina regions may be provided to permit movement of the electrical contacts. A spacer material 404 gives a level surface across the area around and including the sensor.

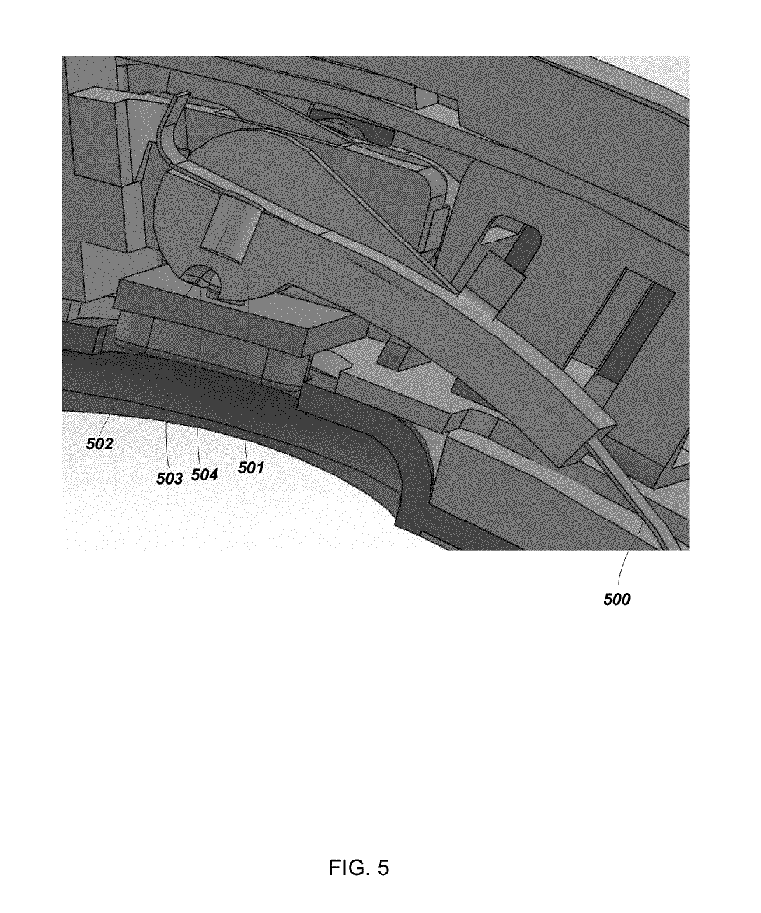

FIG. 5 shows a schematic diagram of a mounting system to allow an EEG sensor, such as that described hereinbefore, to be retracted when not required. The arm 500 of the EEG sensor is mounted on a pivoting mount 501. Mount 501 is configured to pivot about an axis 502 together with the arm 500. As the mount rotates anti-clockwise the arm 500 also rotates and the sensor contacts at the distal end of arm 500 are lifted towards the body of the device in which the sensor is mounted. Sliding switch 503 engages with a recess 504 on the mount 501 and is used by the user to move the sensors towards or away from the device.

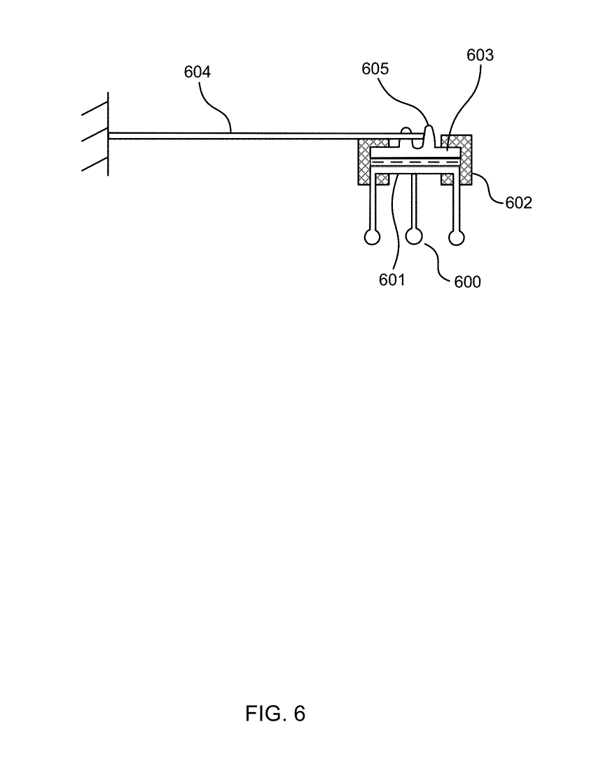

FIG. 6 shows a schematic diagram of a further mounting system for an EEG sensor. Electrical contacts 600 are mounted to, or form unitarily with, a bobbin 601, which is held by a resilient mounting 602. The resilient mounting 602 connects the bobbin and contacts 600, 601 to a mounting component 603. Mounting component 603 is connected to resilient arm 604 by a ball joint 605. Ball joint 605 provides a pivot connection between the mounting component 603 (and hence bobbin 601 and contacts 600) and resilient arm 604. Any pivoting connection that provides a pivotable connection in 1 or 2 dimensions may be utilised in place of the ball joint 605.

A low-friction surface is provided between bobbin 601 and mounting component 603 to allow lateral movement of the bobbin 601 relative to mounting component 603. This surface may be provided as described hereinbefore. The resilient mounting 602 may be formed of any material which holds the bobbin 601 against the mounting component 603, but which is sufficiently flexible to allow sufficient lateral movement, for example rubber or silicone. The mounting component can also be formed of any appropriate material, with particular examples being nylon, Teflon, or other low friction rigid material.

The resilient arm 604 is comparable to the arms described for the examples set out hereinbefore and may use comparable materials and dimensions. Resilient arm 604 is mounted to the body of the device as described hereinbefore.

As described hereinbefore, the bobbin 601 can move laterally to allow movement of the contacts in the lateral plane, which the resilient arm 604 allows vertical movement of the contacts. The ball joint 605 allows pivoting movement of the contacts 600 to accommodate different geometries of the surface which the electrical contacts 600 make contact with, typically the head.

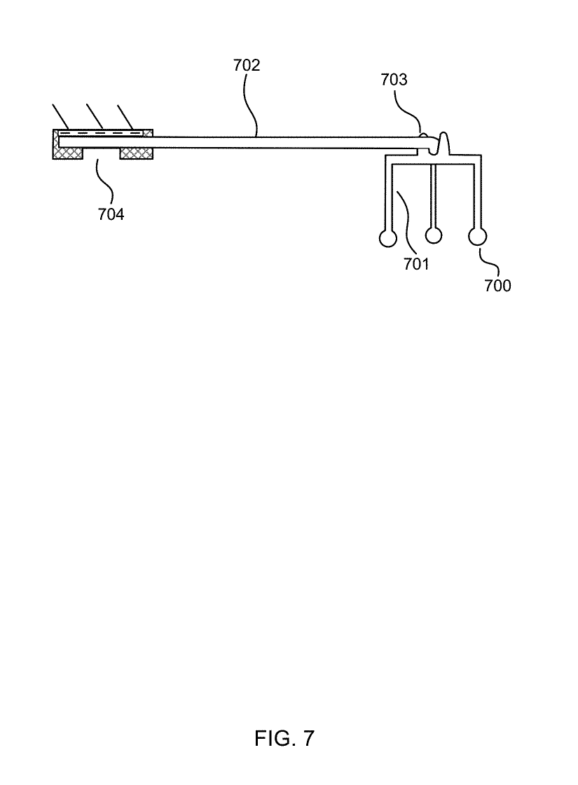

FIG. 7 shows a variation on the arrangement of FIG. 6. The contacts 700 and bobbin 701 are attached directly to the resilient arm 702 by a ball joint 703 which allows pivoting movement between the bobbin 701 and resilient arm 702. Any type of joint may be utilised which provides a pivoting movement in one or more dimension. The resilient arm 702 is mounted to the body of the device in which the sensor is mounted by a joint 704 which allows lateral movement of the resilient arm relative to the body the device. For example, the joint 704 may be provided with a through-slot in which the arm is mounted and which allows the arm to slide along its longitudinal axis.

Various examples of EEG sensors suitable for sensing signals on the scalp or other regions have been described. The sensors are intended to address the difficulties of prior art sensors by maintaining the sensor in a static location on the user's skin, thus avoiding the generation of artefact signals caused by movement. A mechanism to allow retraction of an EEG sensor, for example to avoid damage when not in use, has also been described.

Any range or device value given herein may be extended or altered without losing the effect sought, as will be apparent to the skilled person.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method blocks or elements identified, but that such blocks or elements do not comprise an exclusive list and a method or apparatus may contain additional blocks or elements.

The steps of the methods described herein may be carried out in any suitable order, or simultaneously where appropriate. Additionally, individual blocks may be deleted from any of the methods without departing from the spirit and scope of the subject matter described herein. Aspects of any of the examples described above may be combined with aspects of any of the other examples described to form further examples without losing the effect sought. It will be understood that the above description of a preferred embodiment is given by way of example only and that various modifications may be made by those skilled in the art. Although various embodiments have been described above with a certain degree of particularity, or with reference to one or more individual embodiments, those skilled in the art could make numerous alterations to the disclosed embodiments without departing from the scope of this invention.

An example of an EEG sensor may be a dry EEG sensor, comprising: at least one electrical contact for contacting the skin of a user, each electrical contact being connected to electrical circuitry to process any detected signals. The at least one electrical contact may be mounted in a flexible layer mounted on a part of a device in which the EEG sensor is mounted. The flexible layer may permit movement in all directions with the user's skin when the user's skin moves relative to the device/The flexible layer may support the electrical contact to press the electrical contacts against the skin of the user in use of the device. The electrical contacts may pass through the flexible layer. A void may exist between the electrical contacts and the device. The flexible layer may be formed from silicone. The flexible layer may be provided with concertina regions to permit movement of the electrical contacts. The EEG sensor may be a dry EEG sensor. The flexible layer may be formed from silicone and be provided with concertina regions to permit movement of the electrical contacts. The at least one electrical contact may be mounted in a bobbin. The flexible layer is formed from silicone and is provided with concertina regions to permit movement of the electrical contacts, and wherein the at least one electrical contact is mounted in a bobbin.

Another example of a dry EEG sensor, comprises: at least one elongate electrical contact for contacting the skin of a user, each electrical contact being connected to electrical circuitry to process any detected signals; wherein the at least one electrical contact is mounted in a flexible layer mounted on a part of a device in which the EEG sensor is mounted, wherein the flexible layer permits movement in all directions with the user's skin when the user's skin moves relative to the device, wherein the flexible layer supports the electrical contact to press the electrical contacts against the skin of the user, wherein the electrical contacts pass through the flexible layer, wherein a resilient material is positioned between the at least one electrical contact and a part of a device in which the EEG sensor is mounted; and wherein a low-friction material is positioned between the at least one electrical contact and the resilient material. The EEG sensor may be sufficiently elongate to sense signals on a scalp and reaches through hair on a user's head to make contact with skin. The device may be a set of headphones, and when the headphones are worn by a user, an example EEG sensor as described herein may contact a region of a user's ear. A barrier layer may surround the at least one electrical contact through which the at least one electrical contact protrudes to contact the user's ear.

A set of headphones may include at least one EEG sensor as described herein. At least one EEG sensor may be mounted on the set of headphones to contact skin in the region of a user's ear when the set of headphones is worn.

Another example of a electroencephalography EEG sensor is mounted in a set of headphones. The example EEG sensor may comprise: a flexible layer (402) which permits movement in all directions; at least one elongate electrical contact for contacting the skin of a user, wherein the at least one electrical contacts are mounted in the flexible layer, and the flexible layer provides support for the at least one electrical contacts, wherein in use the EEG sensor contacts a region of an ear of the user.

Another example of a dry EEG sensor which may be mounted in a set of headphones comprises: at least one electrical contact for contacting the skin of an ear of a user; a resilient material positioned between the at least one electrical contact and part of a set of headphones in which the EEG sensor is mounted; and a low-friction material positioned between the at least one electrical contact and the resilient material.

The example EEG sensor may further comprise a barrier layer surrounding the at least one electrical contact. The example EEG sensor may further comprise a bumper within the resilient layer to prevent excessive movement of the at least one electrical contact. The resilient layer may be formed of foam. The example EEG sensor may further comprise a barrier layer surrounding the at least one electrical contact, wherein the barrier layer is formed of fabric. The example EEG sensor may further comprise a barrier layer surrounding the at least one electrical contact, wherein the barrier layer is formed of silicone. The at least one electrical contact may be mounted in a bobbin.

1. A dry EEG sensor, comprising
at least one electrical contact for contacting the skin of a user, each electrical contact being connected to electrical circuitry to process any detected signals;
wherein the at least one electrical contact is mounted in a flexible layer mounted on a part of a device in which the EEG sensor is mounted, wherein the flexible layer permits movement in all directions with the user's skin when the user's skin moves relative to the device,
wherein the flexible layer supports the electrical contact to press the electrical contacts against the skin of the user,
wherein the electrical contacts pass through the flexible layer and a void exists between the electrical contacts and the device.

2. A dry EEG sensor as claimed in claim 1, wherein the flexible layer is formed from silicone.

3. A dry EEG sensor as claimed in claim 1, wherein the flexible layer is provided with concertina regions to permit movement of the electrical contacts.

4. A dry EEG sensor according to claim 1, wherein the flexible layer is formed from silicone and is provided with concertina regions to permit movement of the electrical contacts.

5. A dry EEG sensor according to claim 1, further comprising a barrier layer surrounding the at least one electrical contact.

6. A dry EEG sensor according to claims 1, wherein the at least one electrical contact is mounted in a bobbin.

7. A dry EEG sensor according to claim 1, wherein the flexible layer is formed from silicone and is provided with concertina regions to permit movement of the electrical contacts, and wherein the at least one electrical contact is mounted in a bobbin.

8. A dry EEG sensor as claimed in claim 1, wherein in the void between the electrical contacts and the device, a resilient material is positioned between the at least one electrical contact and a part of a device in which the EEG sensor is mounted; and
wherein a low-friction material is positioned between the at least one electrical contact and the resilient material.

9. A dry EEG sensor according to claim 1, wherein at least one EEG sensor is sufficiently elongate to sense signals on a scalp and reaches through hair on a user's head to make contact with skin.

10. A dry EEG sensor according to claim 1, wherein the device is a set of headphones, and in use at least one EEG sensor contacts a region of a user's ear.

11. A dry EEG sensor according to claim 1, further comprising a barrier layer surrounding the at least one electrical contact through which the at least one electrical contact protrudes to contact the user.

12. A set of headphones in which at least one EEG sensor according to claim 1 is mounted.

13. A electroencephalography EEG sensor mounted in a set of headphones, comprising:
a flexible layer which permits movement in all directions; and
at least one elongate electrical contact for contacting the skin of a user,
wherein the at least one electrical contacts are mounted in the flexible layer and the flexible layer provides support for the at least one electrical contacts,
wherein in use the EEG sensor contacts a region of an ear of the user.

14. An EEG sensor mounted in a set of headphones, comprising
at least one electrical contact for contacting the skin of an ear of a user;
a resilient material positioned between the at least one electrical contact and part of a set of headphones in which the EEG sensor is mounted; and
a low-friction material positioned between the at least one electrical contact and the resilient material.

15. An EEG sensor according to claim 14, further comprising a barrier layer surrounding the at least one electrical contact.

16. An EEG sensor according to claim 14, further comprising a bumper within the resilient layer to prevent excessive movement of the at least one electrical contact.

17. An EEG sensor according to claim 14, wherein the resilient layer is formed of foam.

18. An EEG sensor according to claim 14, further comprising a barrier layer surrounding the at least one electrical contact, wherein the barrier layer is formed of fabric.

19. An EEG sensor according to claim 14, further comprising a barrier layer surrounding the at least one electrical contact, wherein the barrier layer is formed of silicone.

20. An EEG sensor according to claims 14, wherein the at least one electrical contact is mounted in a bobbin.
